(19) United States
(12) Patent Application Publication
(10) Pub. No.: US 2024/0026335 A1
(43) Pub. Date: Jan. 25, 2024

(54) METHOD OF FIXATING A TISSUE SAMPLE

(71) Applicant: Imam Abdulrahman Bin Faisal University, Dammam (SA)

(72) Inventors: Khaled Fikry SALAMA, Dammam (SA); Amal Abdullah ALODAINI, Dammam (SA)

(73) Assignee: Imam Abdulrahman Bin Faisal University, Dammam (SA)

(21) Appl. No.: 17/869,074

(22) Filed: Jul. 20, 2022

(51) Int. Cl. C12N 15/10; G01N 1/30

(52) U.S. Cl. CPC C12N 15/1003; G01N 1/30; G01N 2001/302; G01N 2001/305

(57) ABSTRACT

A method of fixating a tissue sample including: treating the tissue sample with a nano-tissue fixative solution to form a fixated tissue sample. A composition of nucleic acids in the fixated tissue sample is not altered. The nano-tissue fixative solution includes acetic acid, at least one alcohol, chloroform, titanium dioxide nanoparticles, zinc oxide nanoparticles, and silver nanoparticles.

### BACKGROUND

### Technical Field

The present disclosure is directed to a method of fixating a tissue, specifically with a nano-tissue fixative solution, the fixated (preserved) tissue formed by the method and a method for preparing a nano-tissue fixative solution for a fixation process.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Conventional tissue fixation is a physiochemical reaction involving gradual diffusion of a fixative solution into tissues. It is an initial step in tissue specimen evaluation and aids in preservation of the tissue's cellular architecture during processing. Fixation also safeguards a cells composition, as the fixation preserves the integrity of cellular bioactive moieties (for example, proteins, carbohydrates, and nucleic acids) to be subsequently examined by different histologic and molecular techniques, which typically require immunohistochemical staining.

Fixative solutions should be safe, affordable, preserve cellular morphology, not modify the specimen cellular composition and the chemical moietie's reactivity that can affect subsequent detection, and be compatible with immunohistochemical stains. However, a fixative solution with these properties has not yet been found.

Currently, formalin is used as a “gold standard” for tissue fixatives. Formalin maintains a tissue's chemical and cellular antigenicity by forming covalent bonds between biological macromolecules. A composition that contains 10% formalin (4% formaldehyde), diluted in water or in a phosphate buffered solution, is considered as a fixative of choice in routine histopathological preparations to preserve tissue specimens for a long period at a reasonable cost. As a result, formalin-fixed paraffin embedded (FFPE) tissues are now the most widely used histologic prepared tissues in the world. Although formalin is widely used as a fixative, there are also a few challenges. Fixation by formalin occurs through protein cross-linking and protein folding, which can decrease total availability of binding sites that can bind to antibodies and diminish immunoreactivity, especially, if the specimen is over fixed. To some extent, the present limitation is reversed by antigen retrieval methods (e.g., enzymatic or heat induced) to restore normal protein folding and improve the accessibility of binding sites on the fixed tissue. However, such process adds more steps during the daily work in immunohistochemical staining and requires a high-level of optimization which can overall increase the specimen turn-around time.

Moreover, formalin has major health related adverse effects. Anatomists, histologists, pathologists, medical students, and embalmers who are frequently exposed to the formalin are at risk for eye, upper respiratory tract, or dermal irritation. Very high levels can cause pulmonary edema, hemorrhage, and death in laboratory animals. Formaldehyde has been classified as a human carcinogen by the International Agency for Research on Cancer (IARC). As a result, formalin exposure needs to be monitored and kept under certain levels. Also, formalin and formalin treated tissue cannot be disposed of in general sewage system and require additional treatment which adds to the cost. Therefore, there has been research into fixative solutions which do not include formaldehyde or any aldehyde compounds.

CN106359367A discloses an aldehyde-free specimen tissue preservation solution, comprising, methanol, ethanol, butanol, hexanol, glycerin, potassium nitrate, phenylformic acid, picric acid, isothiazolone, and muskiness. However, the solution includes chemicals which are considered hazardous, and it was not tested for compatibility with immunohistochemical stains.

CN110631881A discloses a preparation method of a paraffin section of human fatty tissue comprising fixating in an aldehyde-free specimen tissue preservation solution, Carnot's fixing liquid, composed of 60% ethanol, 30% chloroform and 10% glacial acetic acid, and ferric chloride. However, it was not tested for compatibility with immunohistochemical stains.

Perry et. al. [J Histochem Cytochem, 2016, 64, 425] discusses an aldehyde-free fixative solution made of phosphate buffered 70% ethanol. However, the solution is not shown to have any antimicrobial properties.

Hostein et. al. [Diagn Mol Pathol, 2011, 20,52] discusses the use of an alcohol based aldehyde-free fixative solution capable of preserving nucleic acids. However, the solution is not shown to have any antimicrobial properties, requires a large volume of fixative solution, and a long tissue fixation time.

Nanoparticles are generally used worldwide with large-scale applications in various fields, such as medicine. Many metal nanoparticles possess unique properties such as small size, tunable size, a high surface area to volume ratio, biocompatibility, and antimicrobial properties. These properties provide the potential for nanoparticles to be incorporated in fixative solutions, resulting in a “nano-tissue fixative solution”. The small size of the nanoparticles can accelerate penetration into tissue over a large surface area of the tissue and cells, thereby requiring less solution, and less fixation time, resulting in lower costs.

In light of the above, formalin and aldehyde free fixative solutions suffer from one or more drawbacks hindering their adoption. Accordingly, it is an object of the present disclosure to provide a nano-tissue fixative solution for fixating a tissue sample. It is another object of the present disclosure to provide an aldehyde free and hazardous chemical free nano-tissue fixative solution. It is another object of the present disclosure to provide a nano-tissue fixative solution which is compatible with immunohistochemical stains. It is another object of the present disclosure to provide a nano-tissue fixative solution which can be used to fixate tissues in a reduced amount of time and with a reduced amount of fixative solution. It is another object of the present disclosure to provide a nano-tissue fixative solution with antimicrobial properties.

### SUMMARY

In an exemplary embodiment, a method of fixating a tissue sample is described. The method includes treating the tissue sample with a nano-tissue fixative solution to form a fixated tissue sample. A composition of nucleic acids in the tissue sample is not altered. The nano-tissue fixative solution includes acetic acid, at least one alcohol, chloroform, titanium dioxide nanoparticles, zinc oxide nanoparticles, and silver nanoparticles.

In some embodiments, the nano-tissue fixative solution includes 50-70 volume percent (v %) alcohol, 5-15 v % acetic acid, and 15-25 v % chloroform, based on a total volume of the alcohol, acetic acid, and chloroform.

In some embodiments, the nano-tissue fixative solution includes 0.1-5% weight per volume (w/v) titanium dioxide nanoparticles, 0.1-5% w/v zinc oxide nanoparticles, and 0.1-5% w/v silver nanoparticles, based on a total volume of the alcohol, acetic acid, and chloroform.

In some embodiments, the alcohol is at least one selected from the group consisting of methanol, ethanol, and isopropanol.

In some embodiments, the titanium dioxide nanoparticles, zinc oxide nanoparticles, and silver nanoparticles have a substantially spherical shape.

In some embodiments, the titanium dioxide nanoparticles have an average size of 10-50 nanometers (nm).

In some embodiments, the zinc oxide nanoparticles have an average size of 10-50 nm.

In some embodiments, the silver nanoparticles have an average size of 30-100 nm.

In some embodiments, the titanium dioxide nanoparticles, zinc oxide nanoparticles, and silver nanoparticles are agglomerated in the nano-tissue fixative solution.

In some embodiments, the agglomerates are at least 100 nm in size.

In some embodiments, the fixated tissue sample is further stained with at least one stain selected from the group consisting of a hematoxylin and eosin stain, a reticulin stain, a trichrome stain, a periodic acid-schiff stain, a desmin stain, a thyroid transcription factor-1 (TTF1) stain, a cluster of differentiation 3 (CD3) stain, and a Paired-box gene 8 (PAX8) stain.

In some embodiments, there is no bacterial growth on the fixated tissue sample after at least 24 hours.

In some embodiments, the treating of the tissue sample is for 1-8 hours.

In some embodiments, the treating of the tissue sample is for 1-3 hours.

In some embodiments, a ratio of a volume of the tissue sample to a volume of the nano-tissue fixative solution is 1 to 1-20.

In some embodiments, a ratio of a volume of the tissue sample to a volume of the nano-tissue fixative solution is 1 to 1-10.

In some embodiments, a purity of ribonucleic acid (RNA) and deoxy ribonucleic acid (DNA) in the fixated tissue sample is within 5% of a same tissue sample fixated with formalin.

In some embodiments, a quantity of DNA in the fixated tissue sample is higher than a same tissue sample fixated with formalin.

In some embodiments, the nano-tissue fixative solution is made by a method including sonicating a mixture of acetic acid, alcohol, chloroform, titanium dioxide nanoparticles, zinc oxide nanoparticles, and silver nanoparticles for at least 10 minutes. The method further includes exposing the mixture to ultraviolet radiation for at least 20 minutes to form an irradiated solution. The method further includes diluting the irradiated solution with at least double a volume of the mixture with deionized water to form a dilute solution. The method further includes filtering the dilute solution to obtain the nano-tissue fixative solution.

The foregoing general description of the illustrative present disclosure and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is a schematic flow diagram of a method of fixating a tissue sample, according to certain embodiments;

FIG. 1B is a schematic flow diagram of a method of preparing a nano-tissue fixative solution, according to certain embodiments;

FIG. 2 shows microscopic sections, with 10× magnification, of different organs for similar fixation time in an embodiment and 10% neutral buffered formalin (hematoxylin and eosin stain), according to certain embodiments;

FIG. 3 shows microscopic sections, with 10× magnification, of different organs for similar fixation time in an embodiment and 10% neutral buffered formalin with different histochemical stains, according to certain embodiments;

FIG. 4 shows microscopic sections, with 10× magnification, of different organs for similar fixation time in an embodiment and 10% neutral buffered formalin with different immunohistochemical stains following antigen retrieval, according to certain embodiments;

FIG. 5 shows microscopic sections, with 10× magnification, of different organs for similar fixation time in an embodiment and 10% neutral buffered formalin with different immunohistochemical stains with and without antigen retrieval, according to certain embodiments;

FIG. 6 illustrates antimicrobial testing an embodiment and 10% neutral buffered formalin using several dilutions showing no growth after 24 hours incubation, according to certain embodiments;

FIG. 7 is microscopic sections, with 10× magnification, of different organs after 1-4 hours fixation time in an embodiment and 10% neutral buffered formalin illustrating effect of reduced tissue fixation time, according to certain embodiments;

FIG. 8 is microscopic sections, with 10× magnification, of different organs after 24-hours fixation time in an embodiment and 10% neutral buffered formalin illustrating effect of low fixative to tissue volume ratio (<15-20:1), according to certain embodiments;

FIG. 9A is a graphical representation showing ribonucleic acid (RNA) purity in an embodiment of the fixated tissue versus 10% neutral buffered formalin fixated tissue in different conditions, according to certain embodiments;

FIG. 9B is a graphical representation showing RNA quantity in an embodiment of the fixated tissue versus 10% neutral buffered formalin fixated tissue in different conditions, according to certain embodiments;

FIG. 9C is a graphical representation showing deoxyribonucleic acid (DNA) purity in an embodiment of the fixated tissue versus 10% neutral buffered formalin fixated tissue in different conditions, according to certain embodiments;

FIG. 9D is a graphical representation showing DNA quantity in an embodiment of the fixated tissue versus 10% neutral buffered formalin fixated tissue in different conditions, according to certain embodiments; and

FIG. 10 shows transmission electron microscopes (TEM) analysis of the nano-tissue fixative solution shows distributions of TiO₂, ZnO, and silver nanoparticles in the solution, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values there between.

The term nanoparticle refers to a particle that is between 1 and 1,000 nanometers (nm) in size.

The term “aldehyde containing fixative solution”, and variations thereof, refers to a cross-linking type of fixative containing aldehydes. Many examples of cross-linking fixatives, containing aldehydes are commonplace in histology, including formalin, Bouin's, Glyoxal, Zinc-formalin, Acidic-formalin (AFA) and gluteraldehyde.

The term “formalin” refers to commercial solutions of formaldehyde in water commonly used for preservation of biological specimens. Formalin used as a fixative typically is 10% neutral buffered formalin, but other solution concentrations also can be used. Thus, useful formalin fixation concentrations typically range from greater than 0% up to at least 20%, more typically from 5% up to 15%, with certain disclosed working embodiments of the present invention using a 10% neutral buffered formalin solution to fix tissue samples.

The term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, nucleic acids and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

The term “antibody” refers to a polypeptide ligand comprising at least a light chain or heavy chain immunoglobulin variable region which specifically recognizes and binds to an antigen. Antibodies are composed of a heavy and a light chain, each of which has a variable region, termed the variable heavy (VH) region and the variable light (VL) region. Together, the VH region and the VL region are responsible for binding the antigen recognized by the antibody.

Aspects of the present disclosure are directed to a method of fixating tissue samples and preparing a fixative solution for fixating the tissue sample. Particularly, the tissue sample is treated with a nano-tissue fixative solution, as a substitute to formalin, to form a fixated tissue sample. The nano-tissue fixative solution includes at least one alcohol, acetic acid, and chloroform with a nanoparticle mixture of titanium dioxide (TiO₂) nanoparticles, zinc oxide (ZnO) nanoparticles, and silver (Ag) nanoparticles. The nano-tissue fixative solution meets the fixative criteria, as it inhibits autolysis and stabilizes the tissue sample by cross-linking with amino groups of amino acids present in peptides and proteins. Ag, TiO₂ and/or ZnO nanoparticles can act as bridging units between molecules in a tissue sample. Combinations of the Ag, TiO₂ and/or ZnO nanoparticles may form networks that associate with and bridge molecules in the tissue sample, preferably incorporating one or more solvent molecules thereby forming a gel-like matrix.

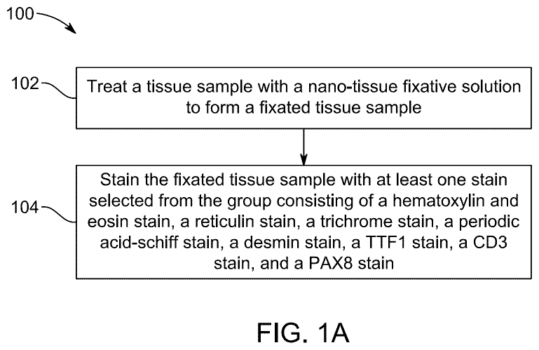

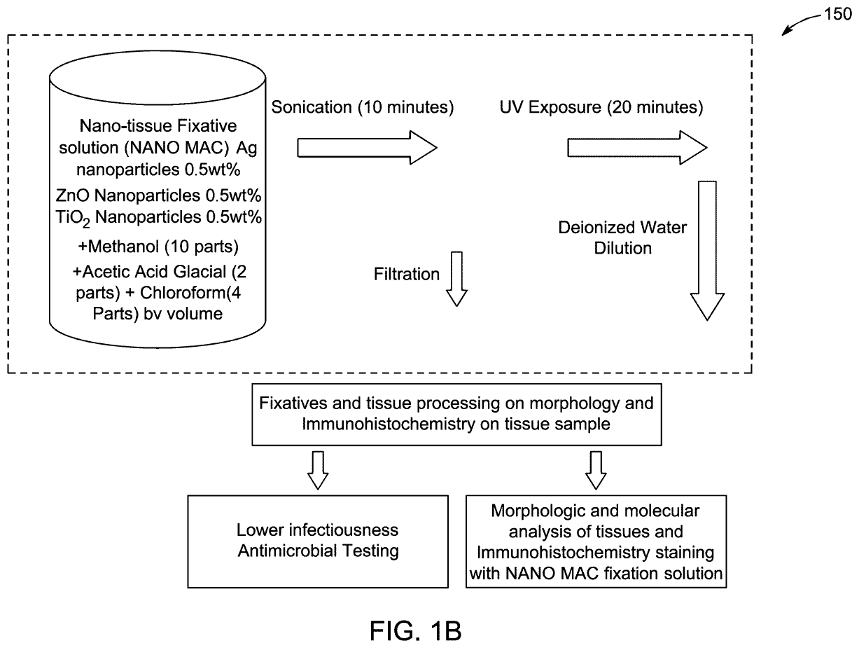

FIG. 1A illustrates a method 100 of fixating a tissue sample. The order in which the method 100 is described is not intended to be construed as a limitation, and any number of the described method steps can be combined in any order to implement the method 100. Additionally, individual steps may be removed or skipped from the method 100 without departing from the spirit and scope of the present disclosure.

At step 102, the method 100 includes treating the tissue sample with a nano-tissue fixative solution to form a fixated tissue sample. In an embodiment, the tissue sample is from any animal. Animal refers to multi-cellular vertebrate organisms, a category that includes, for example, mammals and birds. The term mammal includes both human and non-human mammals. Similarly, the term “subject” includes both human and veterinary subjects, for example, humans, non-human primates, dogs, cats, horses, and cows. In an embodiment, the tissue sample is obtained from a deceased subject. In an embodiment, the tissue is connective tissue, epithelial tissue, muscle tissue, and/or nervous tissue. In an embodiment, the tissue is collected from any part of the subject, including but not limited to the tongue, lips, salivary glands, parotid glands, submandibular glands, sublingual glands, pharynx, esophagus, stomach, small intestine, duodenum, jejunum, ileum, large intestine, cecum, ascending colon, transverse colon, descending colon, sigmoid colon, rectum, liver, gallbladder, mesentery, pancreas, anal canal, nasal cavity, pharynx, larynx, trachea, bronchi, bronchioles, lungs, kidneys, ureter, bladder, urethra, ovaries, fallopian tubes, uterus, cervix, testes, epididymis, vas deferens, seminal vesicles, prostate, bulbourethral glands, pituitary gland, pineal gland, thyroid gland, parathyroid glands, adrenal glands, pancreas, heart, lymph node, bone marrow, thymus, spleen, tonsils, cerebrum, cerebral hemispheres, diencephalon, the brainstem, midbrain, medulla oblongata, cerebellum, eye, cornea, iris, ciliary body, lens, retina, ear, outer ear, earlobe, eardrum, middle ear, ossicles, inner ear, cochlea, olfactory epithelium, mammary glands, skin, subcutaneous tissue, ligaments, or tendons. In an embodiment, the tissue is from a tumor. In an embodiment, the tumor may include but is not limited to sarcomas and carcinomas, including fibrosarcoma, myosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma).

In an embodiment, the nano-tissue fixative solution includes acetic acid, at least one alcohol, and chloroform. In an embodiment, the acetic acid is glacial acetic acid. In some embodiments, the alcohol is at least one selected from the group consisting of methanol, ethanol, and isopropanol. In some embodiments, the alcohol may include, but are not limited to, hexanol, and glycerol. In some embodiments, the at least one alcohol is a combination of methanol and isopropanol. In an embodiment, the alcohol is methanol. In an embodiment, the nano-tissue fixative solution of the present disclosure includes acetic acid, methanol, and chloroform, and is identified as “NANO MAC” herein.

In an embodiment, the nano-tissue fixative solution also includes titanium dioxide nanoparticles, zinc oxide nanoparticles, and silver nanoparticles. In some embodiments, each of the titanium dioxide nanoparticles, zinc oxide nanoparticles, and silver nanoparticles have a substantially spherical shape. In some embodiments, each of the titanium dioxide nanoparticles, zinc oxide nanoparticles, and silver nanoparticles may individually have a spherical, rod, oval, cubic, triangular, star, needle, octahedral, hexagonal. pentagonal, flower, platelet, or cylindrical shape. In an embodiment, the titanium dioxide nanoparticles, the zinc oxide nanoparticles, and the silver nanoparticles are agglomerated in the nano-tissue fixative solution. In some embodiments, the agglomerates are at least 100 nanometers (nm) in size, preferably 100 to nm, 500 to 9,000 nm, 1,000 to 8,000 nm, 2,000 to 7,000 nm, 3,000 to 6,000 nm, 4,000 to nm. In an embodiment, the size of the agglomerates refers to its longest dimension. In some embodiments, the titanium dioxide nanoparticles have an average size of 10-50 nm, preferably 15-45 nm, 20-40 nm, 25-35 nm, or approximately 30 nm. In some embodiments, the zinc oxide nanoparticles have an average size of 10-50 nm, preferably 15-45 nm, 20-40 nm, 25-nm, or approximately 30 nm. In some embodiments, the silver nanoparticles have an average size of 30-100 nm, preferably 35-95 nm, 40-90 nm, 45-85 nm, 50-80 nm, 55-75 nm, 60-70 nm, or approximately 65 nm. In an embodiment, the size of the nanoparticles refers to the diameter.

In some embodiments, the nano-tissue fixative solution includes 50-70 volume percent (v %) alcohol, preferably 55-65 v %, or approximately 60 v %, 5-15 v % acetic acid, preferably 7-12 v % or approximately 10 v %, and 15-25 v % chloroform, preferably 18-22 v % or approximately 20 v %, based on a total volume of the alcohol, acetic acid, and chloroform.

In some embodiments, the nano-tissue fixative solution includes 0.1-5% weight per volume (w/v) titanium dioxide nanoparticles, preferably 0.2-4.5% w/v, 0.3-4% w/v, 0.4-3.5% w/v, 0.5-3% w/v, or 1-2% w/v, 0.1-5% w/v zinc oxide nanoparticles, preferably 0.2-4.5% w/v, w/v, 0.4-3.5% w/v, 0.5-3% w/v, or 1-2% w/v, and 0.1-5% w/v silver nanoparticles preferably 0.2-4.5% w/v, 0.3-4% w/v, 0.4-3.5% w/v, 0.5-3% w/v, or 1-2% w/v, based on a total volume of the alcohol, acetic acid, and chloroform. In an embodiment, the titanium dioxide nanoparticles, zinc oxide nanoparticles, and silver nanoparticles are added in equal w % amounts. In an embodiment, the titanium dioxide nanoparticles, zinc oxide nanoparticles, and silver nanoparticles are added in unequal w % amounts. In an embodiment depicted in FIG. 1B, the nano-tissue fixative solution includes methanol (10 parts), acetic acid glacial (2 parts), and chloroform (4 parts) by volume with a nanoparticle mixture of the titanium dioxide nanoparticles, zinc oxide nanoparticles, and silver nanoparticles in 0.5 wt. %.

Referring to FIG. 1B, a method 150 of preparing the nano-tissue fixative solution is illustrated, according to certain embodiments. The order in which the method 150 is described is not intended to be construed as a limitation, and any number of the described method steps can be combined in any order to implement the method 150. Additionally, individual steps may be removed or skipped from the method 150 without departing from the spirit and scope of the present disclosure.

The method 150 includes sonicating a mixture of acetic acid, alcohol, chloroform, titanium dioxide nanoparticles, zinc oxide nanoparticles, and silver nanoparticles for at least 10 minutes, preferably 10-100 minutes, 20-80 minutes, or 40-60 minutes. As used herein, the term ‘sonication’ refers to a process of applying sound energy to agitate particles or discontinuous fibers in a liquid. In an embodiment, the sonication is at a power of 90-130 Watts (W), preferably 100-120 W, or approximately 110 W. In an embodiment, the sonication is at a frequency of 20-40 kHz, preferably 25-35 kHz, or approximately 30 kHz. In an embodiment, sonication is believed to promote interaction between the solvents and nanoparticles in the nano-tissue fixative solution. In an embodiment, during sonication at least one of the nanoparticles reacts with at least one of the solvents, e.g., to functionalize the surface of the nanoparticles. In an embodiment, at least one of the solvents is bonded to the surface of at least one of the nanoparticles, e.g., to form a metal-solvent bond such as a metal-O—C, metal-S—C bond. In an embodiment, the chloroform, alcohol, and/or acetic acid is bonded to the surface of the silver, titanium dioxide, and/or zinc oxide nanoparticles. In an embodiment, the surface of the silver nanoparticles is modified with a ligand to promote interaction with at least one of the solvents, titanium dioxide, and/or zinc oxide nanoparticles. In an embodiment, the surface of the zinc oxide nanoparticles is modified with a ligand to promote interaction with at least one of the solvents, silver, and/or titanium dioxide nanoparticles. In an embodiment, the surface of the titanium dioxide nanoparticles is modified with a ligand to promote interaction with at least one of the solvents, silver, and/or zinc oxide nanoparticles. In an embodiment, the ligand may include but is not limited to cetyltri-methylammonium bromide, hexadecylamine, oleyamine, sodium citrate, 2-mercaptoethanol, acetic acid, acrylic acid, ammonium thiocyanate, ethanedithiol, and polyethylene glycol. In an embodiment, the ligands interact through ligand interdigitation, hydrogen bonding, and/or electrostatic interactions. In an embodiment, the interaction of the solvents and nanoparticles promotes diffusion of the nano-tissue fixative solution in tissue samples. A functionalized metal nanoparticle may permit better interaction to the tissue through a more lipophilic character.

The method 150 further includes exposing the mixture to ultraviolet radiation (UV) for at least 20 minutes to form an irradiated solution. In an embodiment, the UV radiation has a power of 10-40 W, preferably 20-30 W, or approximately 25 W. In an embodiment, at least 50% of the solution is irradiated, preferably 60%, 70%, 80%, 90%, or 100%. In an embodiment, the UV radiation has a wavelength of 10-400 nm, preferably 50-350 nm, 100-300 nm, 150-250 nm, or approximately 200 nm. In an embodiment, the solution is irradiated for 20 mins to 5 hours, preferably 30 minutes to 4 hours, 1-3 hours, or approximately 2 hours. In an embodiment, the silver, zinc oxide and/or titanium dioxide nanoparticles act as photocatalytic sterilizers. In an embodiment, following UV radiation the silver, zinc oxide and/or titanium dioxide nanoparticles absorb the UV light generating an electron and hole. The hole can then oxidize water in the atmosphere to form hydroxyl radicals and the electron can reduce oxygen to form superoxide anions. The hydroxyl radicals and superoxide anions can disrupt the cell walls and replication of viruses and bacteria resulting in cell death.

The method 150 further optionally includes diluting the irradiated solution with at least double a volume of the total mixture, preferably 2 times, 3 times, 4 times, 5 times, 6 times, 7 times, 8 times, 9 times, 10 times, 20 times, 30 times, 40 times, or 50 times the volume of the total mixture, with deionized water to form a dilute solution. As used herein, the term ‘deionized water’ refers to water having most of the ions removed. The method 150 further optionally includes filtering the dilute solution to obtain the nano-tissue fixative solution. In an embodiment, the filtering is to remove any solid particulates from the solution greater than 10 μm in size. In other words, after filtration the nanoparticles are still present in the nano-tissue fixative solution. In an embodiment, the solution is filtered through a filter paper.

At step 102 of the method 100, the tissue sample is treated with a fixative solution under conditions that allow the fixative to diffuse throughout substantially the entire cross section of the sample. This step 102 is conducted using a volume of fixative for a period of time that allows for complete tissue infusion/diffusion. In some embodiments, the tissue sample is treated with a desired volume of the nano-tissue fixative solution. The desired volume of the nano-tissue fixative solution may be defined based on a volume of the sample tissue. In one embodiment, a ratio of a volume of the tissue sample to a volume of the nano-tissue fixative solution is 1 to 1-preferably 1 to 20, 1 to 15, 1 to 10, 1 to 5, 1 to 1. In one embodiment, a ratio of a volume of the tissue sample to a volume of the nano-tissue fixative solution is 1 to 20. In another embodiment, a ratio of a volume of the tissue sample to a volume of the nano-tissue fixative solution is 1 to 10. In an embodiment, less volume of the nano-tissue fixative solution is required to fixate a tissue sample than formalin under the same conditions. In an embodiment, a lower volume of nano-tissue fixative solution is preferable to save costs. Upon selecting the desired volume of the nano-tissue fixative solution based on the volume of the tissue sample, the tissue sample is treated with the nano-tissue fixative solution for a desired duration. In one embodiment, the tissue sample is treated with the nano-tissue fixative solution for 1-48 hours, preferably 5-40 hours, 10-35 hours, 15-30 hours, or 20-25 hours. In one embodiment, the tissue sample is treated with the nano-tissue fixative solution for 1-8 hours. In another embodiment, the tissue sample is treated with the nano-tissue fixative solution for 1-3 hours. In an embodiment, less time is required to fixate a tissue sample when using the nano-tissue fixative solution than when using formalin under the same conditions. In an embodiment, a lower fixation time is preferable as to save costs and receive rapid results. In some embodiments, the tissue is fixated with the nano-tissue fixative solution at an elevated temperature of 20-40° C., preferably 25-35° C. or approximately 30° C.

In an embodiment, no bacterial growth is observed in the fixated tissue sample after 24 hours, preferably 36 hours, 48 hours, or 72 hours. In an embodiment, less than 10% of the surface area of the fixated tissue sample exhibits bacteria growth after 24 hours, preferably less than 5% or less than 1%. In an embodiment, the nano-tissue fixative solution has antimicrobial properties. In an embodiment, the silver nanoparticle's structure penetrates a viral or bacterial cell wall, resulting in membrane structural damage and cell death.

In an embodiment, the tissue sample has a composition of nucleic acids which is not altered when the tissue sample is fixated with the nano-tissue fixative solution. Nucleic acids are defined as deoxyribonucleic acid (DNA) and ribonucleic acid (RNA). In an embodiment, a quantity of DNA in the fixated tissue sample is higher than the same tissue sample fixated with formalin. In an embodiment, a quantity of DNA in the fixated tissue sample is within 5%, preferably 4%, 3%, 2%, or 1% of the tissue sample fixated with formalin. In an embodiment, a quantity of RNA in the fixated tissue sample is higher than the same tissue sample fixated with formalin. In an embodiment, a quantity of RNA in the fixated tissue sample is within 5%, preferably 4%, 3%, 2%, or 1% of the tissue sample fixated with formalin. In an embodiment, a purity of RNA and DNA in the fixated tissue sample is within 5%, preferably 4%, 3%, 2%, or 1% of the tissue sample fixated with formalin. In an embodiment, the nano-tissue fixative solution does not chemically modify the nucleic acids. In an embodiment, the nano-tissue fixative solution does not chemically modify a composition of nucleic acids, carbohydrates, and/or lipids in the tissue sample. In an embodiment, the nano-tissue fixative solution can be used as a less toxic replacement for formalin.

In an embodiment, the nano-tissue fixative solution stabilizes the tissue sample by cross-linking with amino groups of amino acids present in peptides and proteins. In an embodiment, interaction between the acetic acid, methanol, and/or chloroform solvents and the silver, zinc oxide and/or titanium dioxide nanoparticles promotes the diffusion of the nano-tissue fixative solution in tissue samples. In an embodiment, the solvents and nanoparticles interact through hydrogen bonding of groups on the surface of the nanoparticles and —OH groups in the acetic acid and methanol. In an embodiment, the interaction of the nanoparticles with the solvents allows the nanoparticles to be carried (diffuse) throughout the tissue sample. In an embodiment, the silver, zinc oxide and/or titanium dioxide nanoparticles act as bridging molecules with the acetic acid, methanol, and/or chloroform leading to increased cross-linking of the solvents with the tissue sample. In an embodiment, the cross-linking occurs by photo-cross-linking, where the UV light exposure as previously described generates radicals and anions which promote crosslinking with the amino groups present in the tissue sample.

At step 104, the method 100 includes staining the fixated tissue sample with at least one stain. As used herein, the term ‘staining’ refers to a method of imparting color to cells, tissues or microscopic components, so the cells are highlighted and visualized better under a microscope. In an embodiment, the stain is any stain known in the art. In an embodiment, the stain is hematoxylin and eosin stain, a special stain, and/or an immunohistochemical stain. In an embodiment, the stain is selected from the group consisting of a hematoxylin and eosin stain, a reticulin stain, a trichrome stain, a periodic acid-schiff stain, a desmin stain, a thyroid transcription factor-1 (TTF-1) stain, a cluster of differentiation 3 (CD3) stain, and a Paired-box gene 8 (PAX8 stain).

Hematoxylin and eosin stain is the most common stain used in histology for medical diagnosis. It is the combination of two histological stains: hematoxylin and eosin. The hematoxylin stains cell nuclei a purplish blue, and eosin stains the extracellular matrix and cytoplasm pink, with other structures taking on different shades, hues, and combinations of these colors. Pathologists can thereby differentiate between the nuclear and cytoplasmic parts of a cell, and additionally, the overall patterns of coloration from the stain show the general layout and distribution of cells and provides a general overview of a tissue sample's structure.

Special stains include but are not limited to reticulin stain, trichrome stain, periodic acid-schiff stain. Special stains employ a dye or chemical which has an affinity for a particular tissue component. For example, Massons trichrome stain helps to highlight the supporting collagenous stroma in sections from a variety of organs. This helps to determine a pattern of tissue injury. Trichrome will also aid in identifying normal structures, such as connective tissue capsules of organs, the lamina propria of the gastrointestinal tract, and the bronchovascular structures in the lung. Another example includes a reticulin stain which is useful in parenchymal organs such as liver and spleen to outline the architecture. Delicate reticular fibers, which are argyrophilic, can be seen. A reticulin stain occasionally helps to highlight the growth pattern of neoplasms.

Immunohistochemical staining is accomplished with antibodies that recognize the target antigen. Immunohistochemical stains include but are not limited to desmin stain, TTF-1 stain, CD3 stain, and PAX8 stain. For example, the PAX8 stain targets PAX8 and helps to visualize the presence of PAX8 in a tissue to potentially diagnose certain cancers. Antigen retrieval steps may be required with immunohistochemical staining. Tissues that have been preserved with formaldehyde or formalin contain a variety of chemical modifications that can reduce the detectability of proteins in biomedical procedures. An antigen retrieval method is typically used to reduce these chemical modifications and improve image quality of immunohistochemical stained tissues. In an embodiment, a tissue sample fixated with the nano-tissue fixative solution does not require an antigen retrieval step prior to staining with an immunohistochemical stain.

In an embodiment, the fixated tissue sample is submerged in a solution of the stain. In an embodiment, a fixated tissue sample fixated with the nano-tissue fixative solution is compatible with a hematoxylin and eosin stain, a reticulin stain, a trichrome stain, a periodic acid-schiff stain, a desmin stain, a thyroid transcription factor-1 (TTF-1) stain, a cluster of differentiation 3 (CD3) stain, and a Paired-box gene 8 (PAX8 stain).

### Examples

The following examples describe and demonstrate exemplary embodiments of the nano-tissue fixative solution described herein. The examples are provided solely for the purpose of illustration and are not to be construed as limitations of the present disclosure, as many variations thereof are possible without departing from the spirit and scope of the present disclosure.

### Example 1: Nano-Tissue Fixative Solution Preparation

Methanol (10 parts), Acetic Acid Glacial (2 parts), and Chloroform (4 parts) by volume and 0.5 wt. % of each of titanium dioxide (TiO₂) nanoparticles, zinc oxide (ZnO) nanoparticles, and silver nanoparticles were added together to form a solution. The solution was sonicated for minutes and then exposed to ultraviolet (UV) radiation for 20 minutes. It was then diluted with deionized water (1:10 volume by volume) and filtered through a filter paper to form the nano-tissue fixative solution. This composition of the nano-tissue fixative solution is further referred to as NANO MAC.

### Example 2: Nanoparticle Characterization

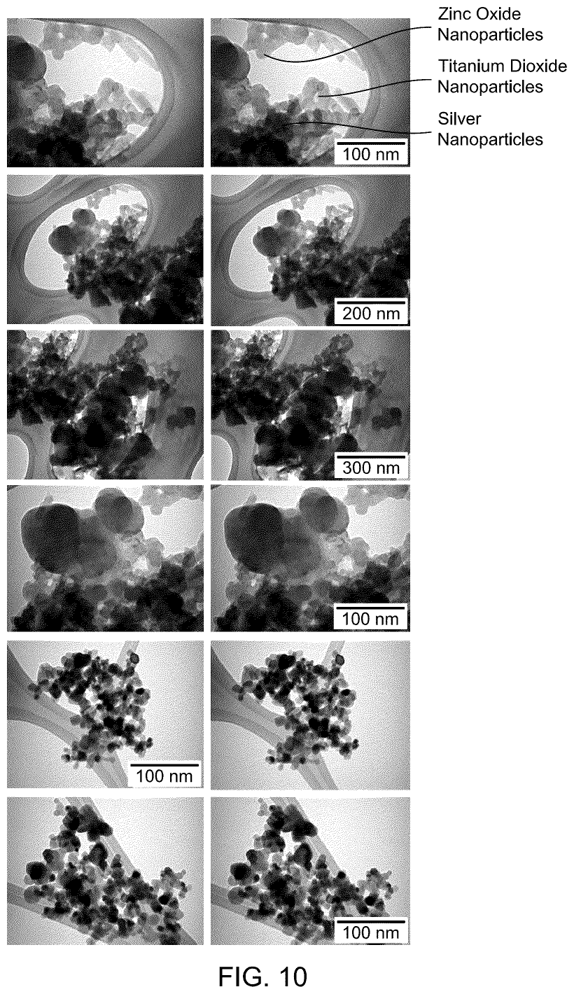

TEM analysis of the nano-tissue fixative solution, as shown in FIG. 10, shows distribution of the titanium dioxide nanoparticles, zinc oxide nanoparticles, and silver nanoparticles therein. The titanium dioxide nanoparticles, zinc oxide nanoparticles, and silver nanoparticles were each spherical in shape and have different diameter sizes. The silver nanoparticles were the darkest in the images and have the largest diameter of 30-100 nm. The titanium dioxide nanoparticles, zinc oxide nanoparticles were both smaller than the silver nanoparticles with a diameter of 10-50 nm. The titanium dioxide nanoparticles, zinc oxide nanoparticles, and silver nanoparticles were agglomerated to form large agglomerates which may be larger than the TEM frame.

### Example 3: Effect of Staining Tissues Following Fixation with NANO MAC

All samples tissue samples were submerged in a solution of the NANO MAC for fixation. Following fixation all samples were embedded with paraffin wax. The paraffin wax was embedded using a tissue embedding machine. Cassette holders and labeled plastic cassettes were used to produce a solidified paraffin block of the specimen. In the embedding machine, paraffin wax was dispensed automatically from a nozzle into a suitably sized mold, which was then placed on a small cool area to allow the wax at the base of the mold to semi-congeal. This allowed easy orientation of the cassette which was placed on top and together they were placed on the cold plate so the paraffin wax could cool quickly, forming crystalline structure. After the paraffin wax had solidified (approximately 5 minutes), the mold was removed, and the block was ready for sectioning.

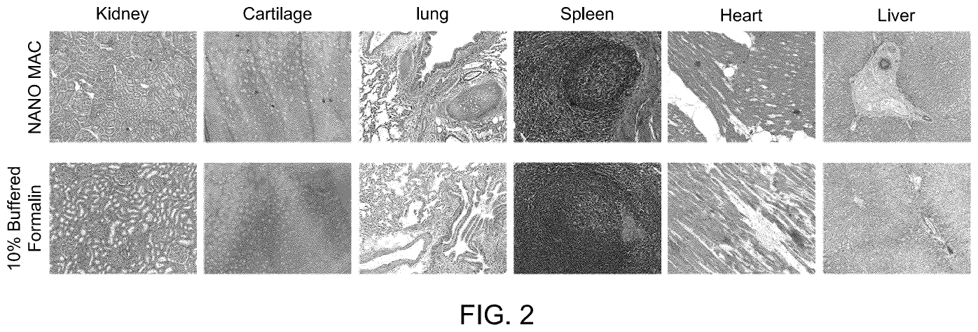

Images of microscopic histological examination from animal samples by routine hematoxylin and eosin stain following fixation with NANO MAC and 10% neutral buffered formalin are compared in FIG. 2. The animal samples included tissues from the kidney, cartilage, lung, spleen, heart, and liver. The images of the hematoxylin and eosin stained NANO MAC fixated tissue samples were comparable to that of the tissue samples fixated with 10% neutral buffered formalin. This indicated that the NANO MAC fixated tissue was compatible with the hematoxylin and eosin stain.

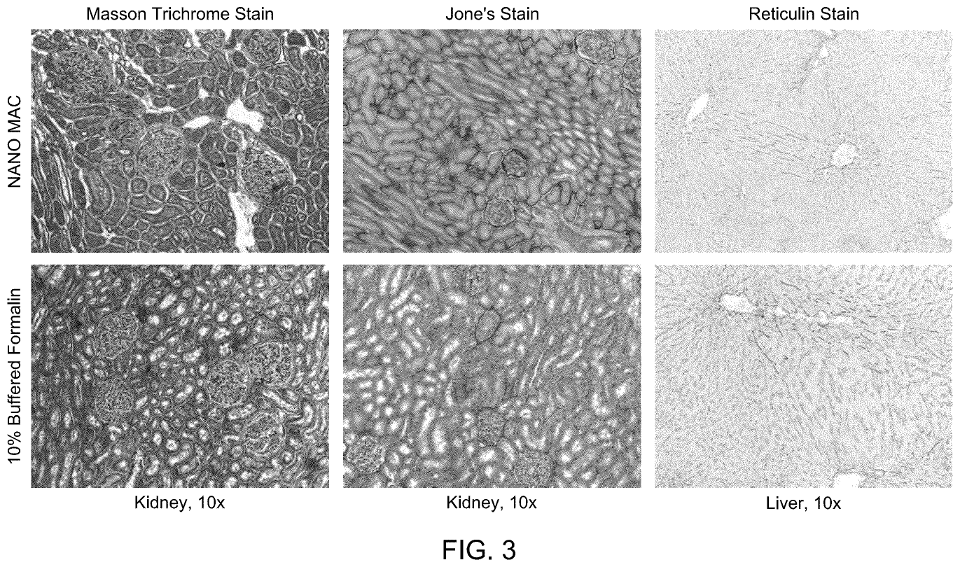

Special stains were also used on NANO MAC fixated kidney and liver tissues and compared with 10% neutral buffered formalin fixated tissues in FIG. 3. The special stains used were Masson trichrome stain, Jones' stain (a periodic acid-schiff stain), and reticulin stain. The images of the Masson trichrome, Jones', and reticulin stained NANO MAC fixated tissue samples were comparable to that of the tissue samples fixated with the 10% neutral buffered formalin fixated tissues. This indicated that the NANO MAC fixated tissue was compatible with the Masson trichrome stain, Jones stain, and reticulin stain.

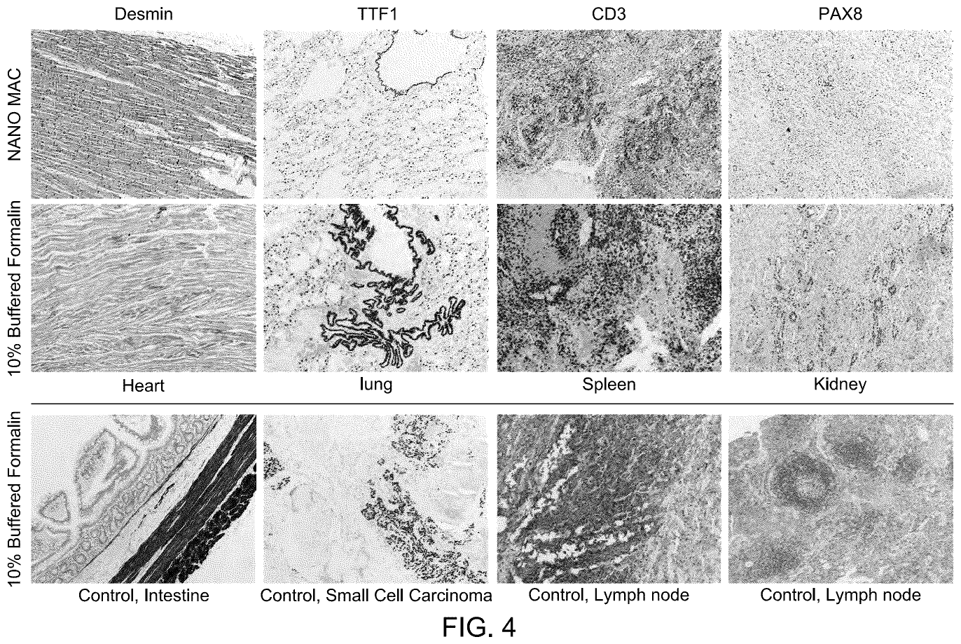

Immunohistochemical stains were also used on NANO MAC fixated heart, lung, spleen, and kidney tissues and compared with 10% neutral buffered formalin fixated tissues in FIG. 4. The immunohistochemical stains used were a desmin stain, a thyroid transcription factor-1 (TTF-1) stain, a cluster of differentiation 3 (CD3) stain, and a Paired-box gene 8 (PAX8 stain). The images of the desmin, TTF1, CD3, and PAX8 stained NANO MAC fixated tissue samples were comparable to that of the tissue samples fixated with the 10% neutral buffered formalin fixated tissues. This indicated that the NANO MAC fixated tissue was compatible with the desmin, TTF1, CD3, and PAX8 stains.

All samples in FIG. 4 had undergone an antigen retrieval step prior to staining except for the desmin stained sample. The Ventana Benchmark Protocol was followed for antigen revival for each sample. Antigen retrieval for each sample was as follows: Desmin: no retrieval was done, TTF-1: slides were warmed up to 95° C. and incubated for 180 min, CD3: slides were warmed up to 95° C. and incubated for 116 min, PAX 8: slides were warmed up to 95° C. and incubated for 64 min.

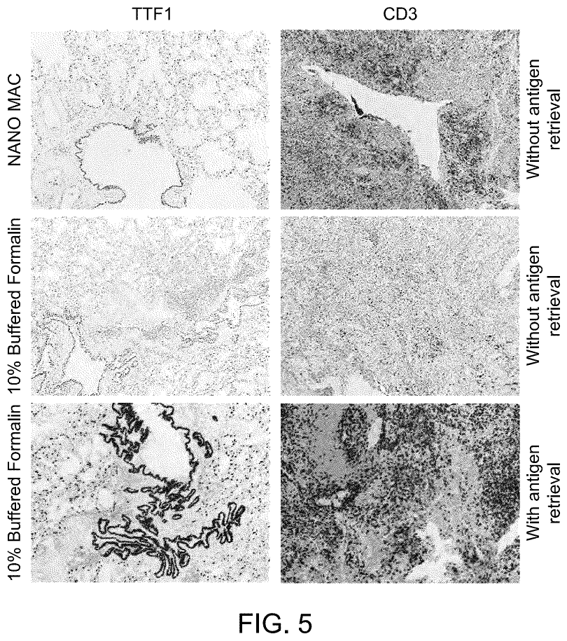

Further, the tissues fixated with NANO MAC showed sufficient reactivity to the immunohistochemical stains without an antigen retrieval step prior to staining, which was not noticed in formalin fixed tissues when this step was eliminated (FIG. 5).

All comparison samples were fixated for the same amount of time.

### Example 4: Antimicrobial Properties of NANO MAC

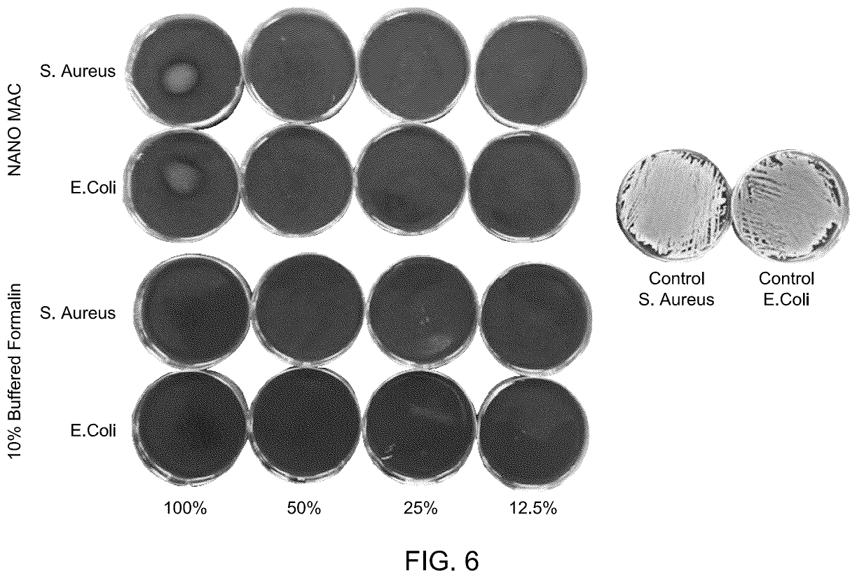

The bacteria S. Aureus and E. Coli exhibited no growth in samples including the NANO MAC solution after 24 hours of incubation. FIG. 6 depicts images of samples with the bacteria and NANO MAC in comparison to samples under the same conditions but with 10% neutral buffered formalin. The samples of NANO MAC and 10% neutral buffered formalin were diluted from 100% to 12.5%. In the dilute samples there was also no bacterial growth. Images of controls of the S. Aureus and E. Coli samples without NANO MAC or 10% neutral buffered formalin grown under the same conditions were shown to have bacterial growth. Therefore, the NANO MAC fixative solution displays antimicrobial properties. This is thought to be due to the presence of silver nanoparticles in the NANO MAC solution. The silver nanoparticles penetrate viral and bacterial cell walls, resulting in membrane structural damage and cell death. Silver nanoparticles are also known to produce free radicals that destroy the viral load.

### Example 5: Effect of Reduced Tissue Fixation Time on NANO MAC Fixative Efficacy

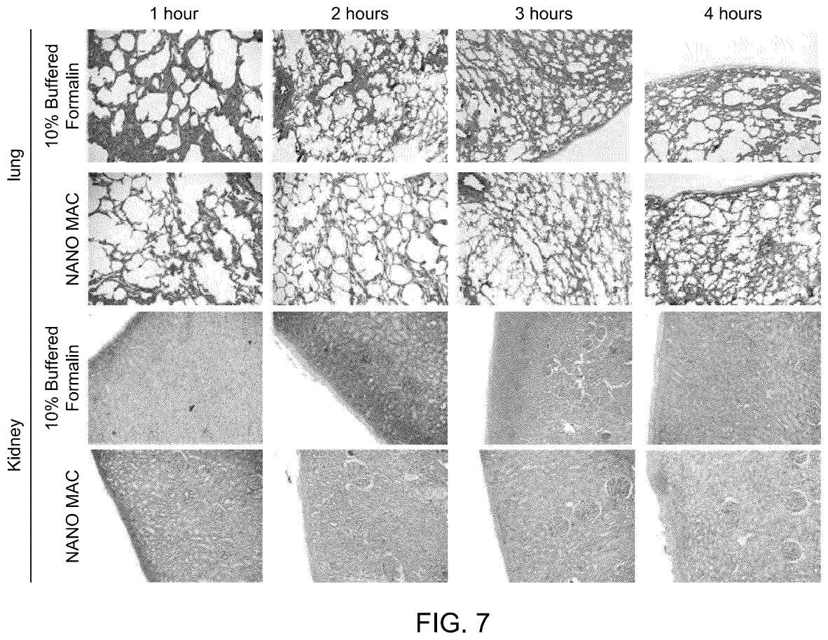

Typically, 6 to 8 hours are required for adequate fixation of tissue in formalin which allows for adequate tissue penetration. Extended periods of fixation are required for large specimens and special tissues like fat, or blood rich organs (for example, lung tissue). Such extended periods can lengthen the overall process of slide preparation and reporting. The quality of fixation was tested in kidney and lung tissue fixed in NANO MAC and 10% neutral buffered formalin for less than recommended fixation time (1-4 hours), but in adequate fixative solution volumes (as shown in FIG. 7). The findings demonstrated better tissue fixation quality for NANO MAC fixated tissue even after only one hour of fixation compared to 10% neutral buffered formalin fixated tissues. The present observation was also valid for tested blood rich organs like lung. Such advantage is useful in situations where rapid initial diagnosis is required.

### Example 6: Effect of Low Fixative to Tissue Volume Ratio on NANO MAC Fixative Efficacy

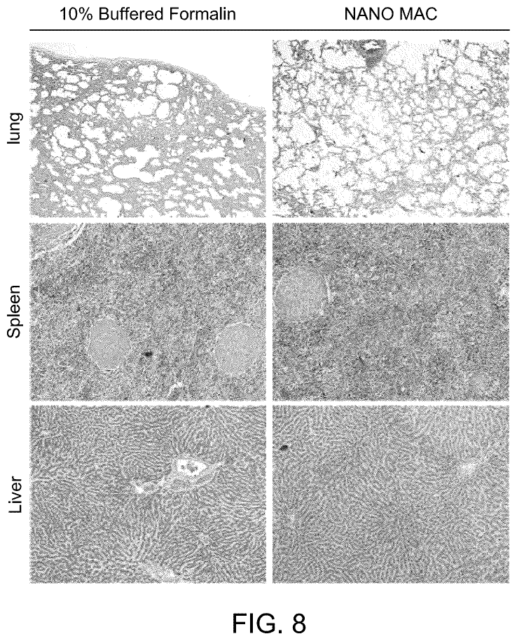

In routine histopathology practice, an adequate amount of fixative is usually considered to be 15 to 20 times the volume of the tissue sample. Also, fixative contaminated with blood or other fluids will be diluted and will not adequately fix tissues, thereby requiring periodic fixative changes to ensure adequate fixation. As such, large specimens (for example, modified radical mastectomies and colectomies) and blood rich organs (example, lung, spleen and liver) require either large fixative volumes to be optimally fixed or periodic renewal of the fixative which can have economic impact in financially limited laboratories. Similar tissue volumes of different organs were fixed in fixative volumes less than the standard recommendation, i.e. less than 15 to times the volume of the tissue sample for 24 hours. Organs fixed in NANO MAC solution showed both better structural preservation and staining than tissues fixed in 10% neutral buffered formalin under similar conditions (as shown in FIG. 8). Such results indicate the potential advantage of NANO MAC as a fixative can be used in smaller volumes and deliver better fixation.

### Example 7: Effect of NANO MAC on the Nucleic Acids, RNA and DNA, Purity and Quantity in NANO MAC Fixated Tissue

The RNA and DNA nucleic acids were extracted from formalin fixed paraffin embedded tissues (FFPE) tissues using the RNeasy FFPE Kit manually and The QIAamp DNA FFPE Tissue Kit on QIAcube machine, respectively. The RNA and DNA purity and quantities were determined using Epoch UV spectrophotometry (Agilent/BioTeck). A ratio of measured spectrophotometric absorbance of a sample at 260 nm compared to a value measured at 280 nm, labeled as the A260/280 ratio, and was used as an assessment of purity for nucleic acid. The ideal ratios are approximately 1.8 and approximately 2 for DNA and RNA, respectively.

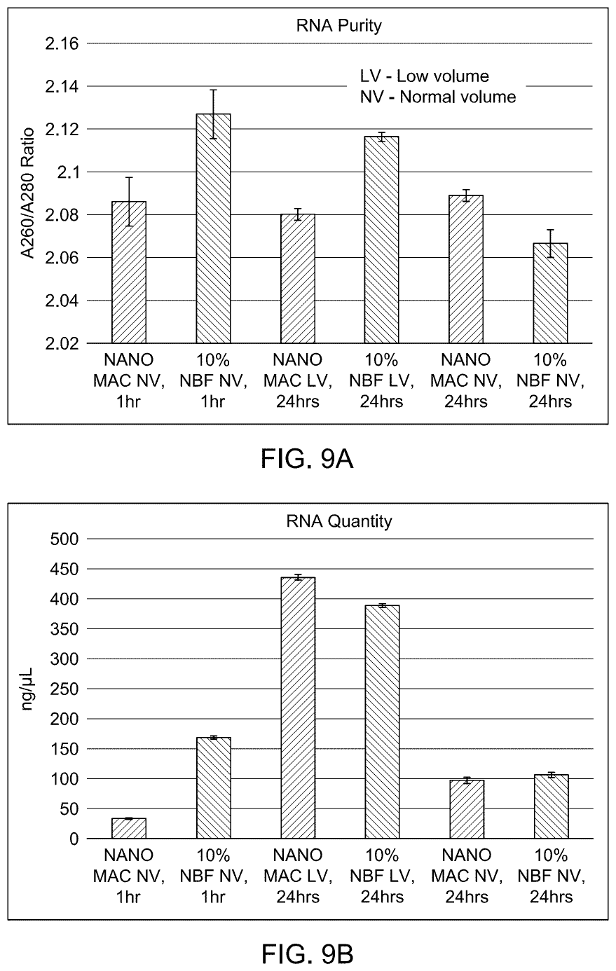

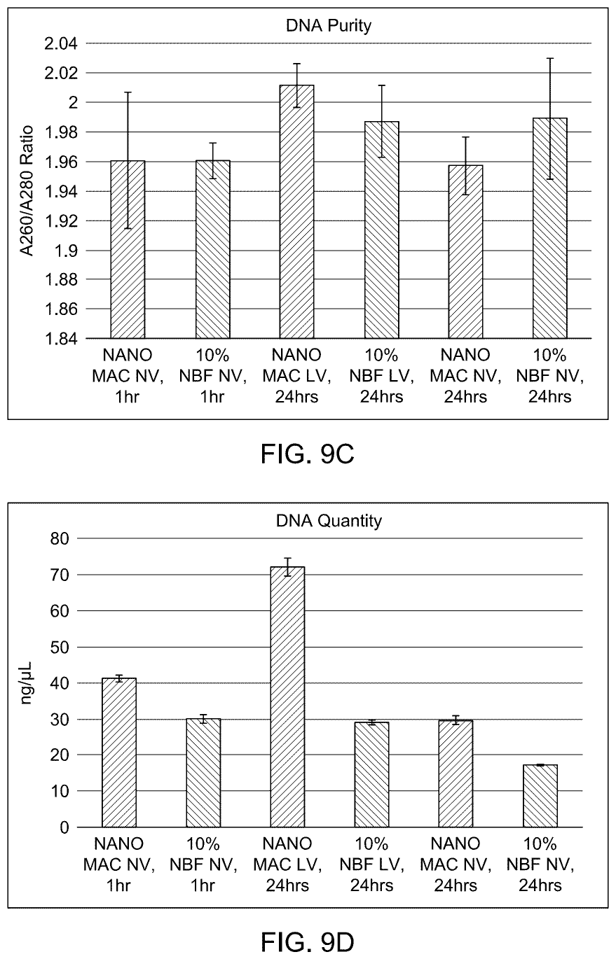

Under standard formalin-fixation and paraffin-embedding procedures, nucleic acids (RNA and DNA) in FFPE samples are chemically modified by formaldehyde. Therefore, nucleic acids isolated from FFPE tissues often have a lower molecular weight than those obtained from fresh or frozen samples. The degree of compromise depends on the type and age of the sample and on the conditions for fixation, embedding, and storage of the sample. The effect of NANO MAC on the RNA and DNA purity and quantity in the NANO MAC fixated tissue in comparison to 10% neutral buffered formalin fixed tissue under similar conditions was investigated, as shown in FIGS. 9A-9D.

Referring to FIG. 9A, RNA purity in the NANO MAC fixated tissue versus 10% neutral buffered formalin in different conditions is illustrated. The different conditions included changes in volume, normal (i.e. fixative solution 15 to 20 times the volume of the tissue sample) and low volume (i.e. less than 15 to 20 times the volume) and changes in fixation time (1 and 24 hours). The A260/A280 ratio of the NANO MAC fixated tissue was 2.06-2.09, irrespective of the condition, which was indicative of pure RNA. Compared to 10% neutral buffered formalin, which varies from 2.07-2.13 under the different conditions.

Referring to FIG. 9B, RNA quantity in the NANO MAC fixated tissue versus 10% neutral buffered formalin fixated tissue in different conditions is illustrated. The different conditions included changes in volume, normal (i.e. fixative solution 15 to 20 times the volume of the tissue sample) and low volume (i.e. less than 15 to 20 times the volume) and changes in fixation time (1 and 24 hours). The RNA quantity extracted from the NANO MAC fixated tissue and 10% neutral buffered formalin fixated tissue was variable (from 25 ng/μg to 440 ng/μg) depending on the preservation condition, however, the RNA quantities in the NANO MAC fixated tissue were comparable to 10% neutral buffered formalin fixated tissues.

Referring to FIG. 9C, DNA purity in the NANO MAC fixated tissue versus 10% neutral buffered formalin in different condition is illustrated. The different conditions included changes in volume, normal (i.e. fixative solution 15 to 20 times the volume of the tissue sample) and low volume (i.e. less than 15 to 20 times the volume) and changes in fixation time (1 and 24 hours). The A260/A280 ratio of the NANO MAC fixated tissue was 1.96 to 2.01, irrespective of the condition, which was indicative of pure DNA. The 10% neutral buffered formalin fixated tissues had similar ratios to that of the NANO MAC fixated tissues.

Referring to FIG. 9D, DNA quantity in the NANO MAC fixated tissue versus 10% neutral buffered formalin fixated tissue in different conditions is illustrated. The different conditions included changes in volume, normal (i.e. fixative solution 15 to 20 times the volume of the tissue sample) and low volume (i.e. less than 15 to 20 times the volume) and changes in fixation time (1 and 24 hours). The DNA quantity (ranging from 29-74 ng/μg) extracted from the NANO MAC fixated tissue was higher under all conditions than 10% neutral buffered formalin fixated tissue.

According to the present disclosure, the nano-tissue fixative solution provides improved chemical, antimicrobial, and fixation properties and is considered as a reliable tool to investigate tissue morphology, and immunohistochemical detection of protein in tissues. The higher immunoreactivity of the nano-tissue fixated tissues requires less processing time which in turn leads to a practical and an economic impact. Further, the nano-tissue fixative solution with the composition thereof, is non-toxic, environment friendly and does not require further treatment prior to disposal, thus reducing the cost of pre-treatment and the lab expenses.

Obviously, numerous modifications and variations of the present disclosure are possible in light of the above teachings. It is therefore to be understood that within the scope of the appended claims, the invention may be practiced otherwise than as specifically described herein.

1. A method of fixating a tissue sample, comprising:
treating the tissue sample with a nano-tissue fixative solution to form a fixated tissue sample;
wherein a composition of nucleic acids in the tissue sample is not altered; and
wherein the nano-tissue fixative solution, comprises:
acetic acid;
at least one alcohol;
chloroform;
titanium dioxide nanoparticles;
zinc oxide nanoparticles; and
silver nanoparticles.

2. The method of claim 1, wherein the nano-tissue fixative solution comprises 50-volume percent (v %) alcohol, 5-15 v % acetic acid, and 15-25 v % chloroform, based on a total volume of the alcohol, acetic acid, and chloroform.

3. The method of claim 2, wherein the nano-tissue fixative solution comprises 0.1-5% weight per volume (w/v) titanium dioxide nanoparticles, 0.1-5% w/v zinc oxide nanoparticles, and 0.1-5% w/v silver nanoparticles, based on a total volume of the alcohol, acetic acid, and chloroform.

4. The method of claim 1, wherein the alcohol is at least one selected from the group consisting of methanol, ethanol, and isopropanol.

5. The method of claim 1, wherein the titanium dioxide nanoparticles, zinc oxide nanoparticles, and silver nanoparticles have a substantially spherical shape.

6. The method of claim 1, wherein the titanium dioxide nanoparticles have an average size of 10-50 nanometers (nm).

7. The method of claim 1, wherein the zinc oxide nanoparticles have an average size of 10-50 nm.

8. The method of claim 1, wherein the silver nanoparticles have an average size of 30-100 nm.

9. The method of claim 1, wherein the titanium dioxide nanoparticles, zinc oxide nanoparticles, and silver nanoparticles are agglomerated in the nano-tissue fixative solution.

10. The method of claim 9, wherein the agglomerates are at least 100 nm in size.

11. The method of claim 1, further comprising:
staining the fixated tissue sample with at least one stain selected from the group consisting of a hematoxylin and eosin stain, a reticulin stain, a trichrome stain, a periodic acid-schiff stain, a desmin stain, a thyroid transcription factor-1 (TTF1) stain, a cluster of differentiation 3 (CD3) stain, and a Paired-box gene 8 (PAX8) stain.

12. The method of claim 1, wherein there is no bacterial growth on the fixated tissue sample after at least 24 hours.

13. The method of claim 1, wherein the treating of the tissue sample is for 1-8 hours.

14. The method of claim 1, wherein the treating of the tissue sample is for 1-3 hours.

15. The method of claim 1, wherein a ratio of a volume of the tissue sample to a volume of the nano-tissue fixative solution is 1 to 1-20.

16. The method of claim 1, wherein a ratio of a volume of the tissue sample to a volume of the nano-tissue fixative solution is 1 to 1-10.

17. The method of claim 1, wherein a purity of ribonucleic acid (RNA) and deoxy ribonucleic acid (DNA) in the fixated tissue sample is within 5% of a same tissue sample fixated with formalin.

18. The method of claim 1, wherein a quantity of DNA in the fixated tissue sample is higher than a same tissue sample fixated with formalin.

19. The method of claim 1, further comprising:
sonicating a mixture of acetic acid, alcohol, chloroform, titanium dioxide nanoparticles, zinc oxide nanoparticles, and silver nanoparticles for at least 10 minutes;
exposing the mixture to ultraviolet radiation for at least 20 minutes to form an irradiated solution;
diluting the irradiated solution with at least double a volume of the mixture with deionized water to form a dilute solution; and
filtering the dilute solution to obtain the nano-tissue fixative solution.
